(19) United States
(12) Patent Application Publication
(10) Pub. No.: US 2021/0145401 A1
(43) Pub. Date: May 20, 2021

(54) Method of Diagnosis with Point-of-Care Ultrasound Devices

(71) Applicant: QISDA CORPORATION, Taoyuan City (TW)

(72) Inventors: Chia-Hsin Lin, Hsinchu County (TW); Yu-Hui Cho, Hsinchu City (TW); Shih-Ting Lin, Hsinchu City (TW)

(21) Appl. No.: 17/005,305

(22) Filed: Aug. 27, 2020

(30) Foreign Application Priority Data
Nov. 19, 2019 (CN) 201911133760.9

(51) Int. Cl. A61B 8/08; A61B 8/00

(52) U.S. Cl. CPC A61B 8/0833; A61B 8/5207; A61B 8/48; A61B 8/461; A61B 8/4444; A61B 8/4427

(57) ABSTRACT

A diagnostic method using a point-of-care ultrasound device (POCUS). The method includes storing at least one operation step corresponding to at least one organ with each operation step corresponding to an ultrasound view, selecting a first operation step from the at least one operation step, displaying a first ultrasound view including a first real-time image and a plurality of first lesions, corresponding to the first operation step, scanning the at least one organ at a first position according to the first operation step to obtain the first real-time image, selecting a first group of lesions from the plurality of first lesions, and generating a first diagnosis according to at least the first group of lesions.

### BACKGROUND OF THE INVENTION

### 1. Field of the Invention

The present invention is related to a method of using ultrasound devices, and more particularly to a method of diagnosis with point-of-care ultrasound devices.

### 2. Description of the Prior Art

A point-of-care ultrasound device (POCUS) is a ultrasound diagnostic device for physicians, especially emergency doctors, to utilize when a patient's diagnosis is unknown. It is an extension to a medical history inquiry and medical image examination. Point-of-care ultrasound refers to the use of a lightweight and portable ultrasound device used to scan directly at or near the patient's bedside in order to find possible lesions in the shortest time. It fits what the emergency care needs. With continually advancement of technology, ultrasound imaging devices have evolved from traditional screens to LCD screens and from heavyweight to lightweight. Even some manufacturers have introduced tablet-type and palm-type ultrasound imaging devices.

The diagnostic process used by POCUS usually cannot be confirmed only by the scanned image of a single part of the human body. Most of the time, the doctor needs to collect clues from the images through different views with the patient's medical history and complaints to confirm the diagnosis. For patients needing urgent care, shortening the diagnostic time and simplifying the diagnostic procedure become extremely important. The present invention standardizes the complicated procedures, and achieves the purpose of allowing doctors to quickly decide on a diagnosis by adding corresponding assistance of medical history, complaints and lesions.

### SUMMARY OF THE INVENTION

The embodiment provides a diagnostic method using a point-of-care ultrasound device (POCUS). The method comprises storing at least one operation step corresponding to at least one organ with each operation step corresponding to an ultrasound view, selecting a first operation step from the at least one operation step, displaying a first ultrasound view including a first real-time image and a plurality of first lesions, corresponding to the first operation step, scanning the at least one organ at a first position according to the first operation step to obtain the first real-time image, selecting a first group of lesions from the plurality of first lesions, and generating a first diagnosis according to at least the first group of lesions.

The embodiment provides a point-of-care ultrasound device (POCUS) comprising a microcontroller configured to execute a diagnostic program, the diagnostic program comprising a plurality of operation steps each corresponding to an ultrasound view, a probe configured to scan at least one organ at a first position to obtain a first real-time image when performing a first operation step of the plurality of operation steps, a display coupled to the microcontroller and configured to display a first ultrasound view corresponding to the first operation step, and a user interface configured to select a first group of lesions from the plurality of first lesions. The first ultrasound view comprises the first real-time image and a plurality of first lesions. The microcontroller generates a first diagnosis according to at least the first group of lesions.

The embodiment provides a diagnostic method using a point-of-care ultrasound device (POCUS). The method comprises storing a plurality of operation steps corresponding to at least one organ with each operation step corresponding to an ultrasound view, selecting a first operation step from the plurality of operation steps, displaying a first ultrasound view including the first real-time image and a plurality of first lesions, corresponding to the first operation step, scanning the at least one organ at a first position according to the first operation step to obtain the first real-time image, selecting a first group of lesions from the plurality of first lesions, selecting a second operation step from the plurality of operation steps, displaying a second ultrasound view including a second real-time image and a plurality of second lesions, corresponding to the second operation step, scanning the at least one organ at a second position according to the second operation step to obtain the second real-time image, selecting a second group of lesions from the plurality of second lesions, and generating a plurality of first diagnoses according to at least the first group of lesions and the second group of lesions. The plurality of first diagnoses has a first level of confidence.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of a point-of-care ultrasound device of an embodiment of present invention.

FIG. 2 is a flowchart of a diagnostic method using the point-of-care ultrasound device of FIG. 1.

FIG. 3 is an example of the diagnostic procedure using the diagnostic method of FIG. 2.

### DETAILED DESCRIPTION

Ultrasound diagnostic devices can be used to diagnose diseases of organs and tissues in the human body, and are generally used in the hospital setting because of their non-invasive and non-radioactive characteristics. Comparing with X-ray and CT scan, the ultrasonic diagnostic device has two benefits. Firstly, it is non-radioactive, and secondly it provides real-time image of the human body. There is no need to wait for film to be processed. It can be used in the cardiovascular field to measure the blood flow rate and disease diagnosis.

A point-of-care ultrasound device (POCUS) is an ultrasound imaging device that can be used by an emergency doctor to diagnose the patient's unknown conditions. POCUS uses a portable ultrasound device to perform ultrasound scan in various hospital rooms, emergency rooms or places such as disaster scenes. When patients have immediate needs, POCUS can help directly solve clinical problems quickly. It is more flexible and efficient than a stationary ultrasound device in a hospital room. The present invention is a diagnostic method using POCUS. By adding corresponding medical history, complaints and lesions, physicians can diagnose the patients more quickly and efficiently.

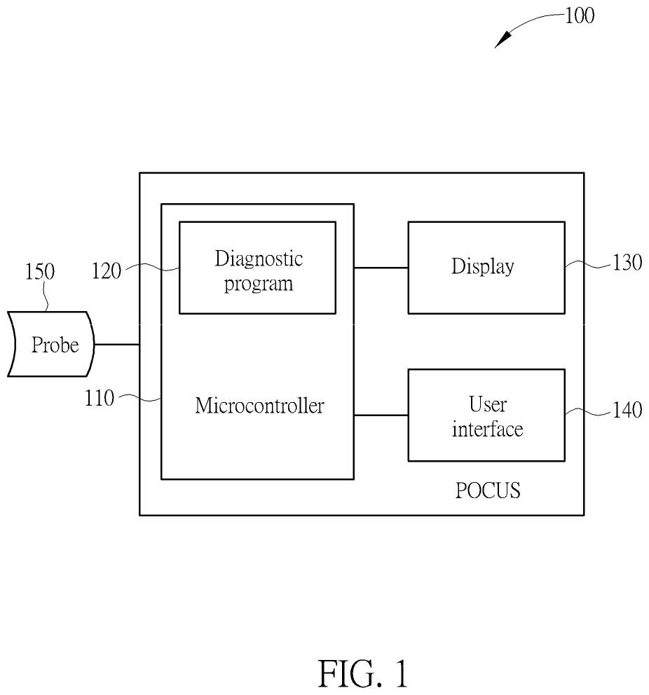

FIG. 1 is a schematic diagram of a point-of-care ultrasound device (POCUS) 100 of an embodiment of present invention. The POCUS 100 includes a microcontroller 110, a display 130, a user interface 140, and a probe 150. The microcontroller 110 is configured to execute a diagnostic program 120. The diagnostic program 120 includes a plurality of operation steps, and each operation step corresponds to an ultrasound view. When an operation step of the plurality of operation steps is performed, the probe 150 is used to scan at least one organ at a position to obtain a real-time image. The display 130 is coupled to the microcontroller 110 to display an ultrasound view corresponding to the operation step. The ultrasound view includes the real-time images and a plurality lesions. The user interface 140 is used to select a group of lesions from the plurality of lesions. The microcontroller 110 generates a diagnosis according to the at least one group of lesions.

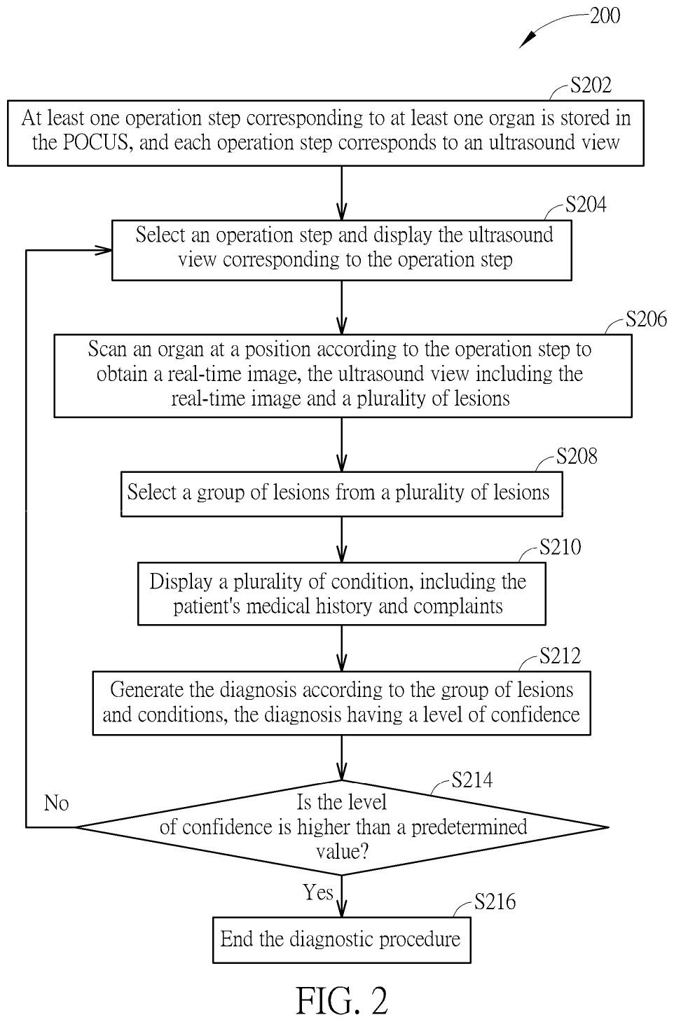

FIG. 2 is a flowchart of a diagnostic method 200 using the point-of-care ultrasound device of FIG. 1. The diagnostic method 200 includes the following steps:

S202: At least one operation step corresponding to at least one organ is stored in the POCUS 100, and each operation step corresponds to an ultrasound view;

S204: Select an operation step and display the ultrasound view corresponding to the operation step;

S206: Scan an organ at a position according to the operation step to obtain a real-time image, the ultrasound view including the real-time image and a plurality of lesions;

S208: Select a group of lesions from a plurality of lesions;

S210: Display a plurality of condition, including the patient's medical history and complaints;

S212: Generate the diagnosis according to the group of lesions and conditions, the diagnosis having a level of confidence;

S214: If the level of confidence is higher than a predetermined value, perform step S216, otherwise perform step S204;

S216: End the diagnostic procedure.

In each iteration of the method 200, in step S206 the scanning position of the probe 150 is different, or the probe 150 can scan from the same position but different angle. In addition, the algorithm used by the POCUS 100 is optimized according to the diagnosis. When confirming the diagnosis, the algorithm is optimized to increase the level of confidence for the next diagnostic process. When denying the diagnosis, the algorithm is optimized to reduce the level of confidence for the next diagnostic process.

For example, the user may define the lesions observed in each ultrasound view in the diagnostic program 120. Each ultrasound view may be different, and the lesions may be changed according to user's input. The diagnostic algorithm in the diagnostic program 120 can be modified according to the user's input, and the corresponding relations between the lesions and the diagnosis include the level of confidence and can be defined in advance according to POCUS related literature. While scanning, each ultrasound view allows the user to check whether the lesions in this ultrasonic view is observed and can record them simultaneously. At the end of the diagnostic process, several possible diagnoses are automatically generated for the user to choose according to the lesions. If the observed lesions, the diagnosis and the sufficient level of confidence can be determined, the user can stop the diagnostic program 120 and generate the diagnostic report.

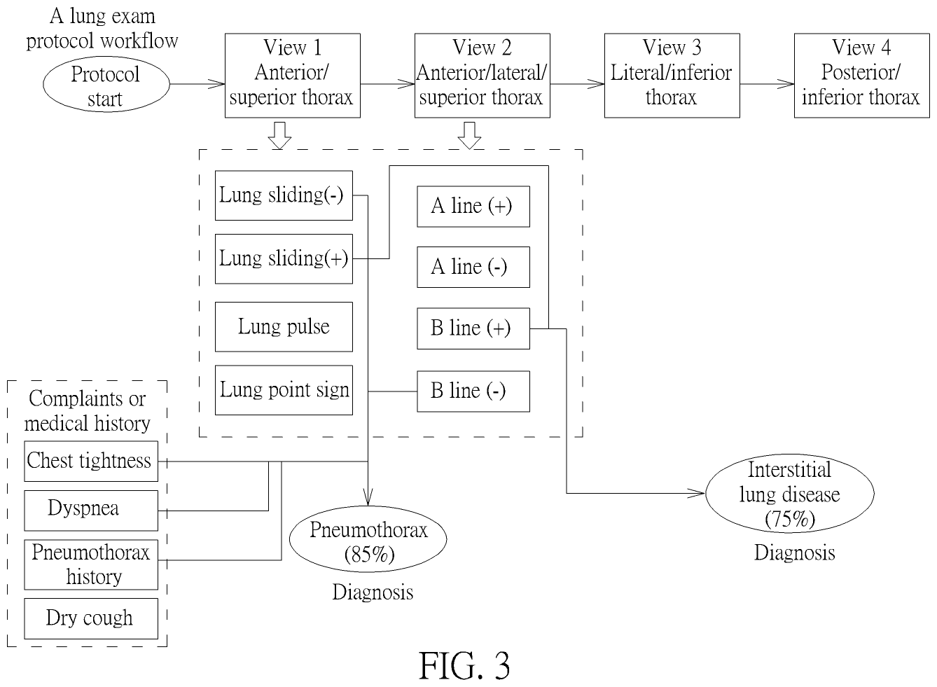

FIG. 3 is an example of the diagnostic procedure using the diagnostic method 200 of FIG. 2. For example, the lungs exam protocol of the POCUS 100 has four operation steps, corresponding to four ultrasound views respectively. View 1 is the anterior/superior thorax, and View 2 is the anterior/lateral/superior thorax. View 3 is the literal/inferior thorax. View 4 is the posterior/inferior thorax.

The user can select the lungs exam protocol when using the POCUS 100. The diagnostic program 120 will first load the operation step corresponding to view 1. Then the user can scan the lungs at the indicated position of the thorax to obtain the real-time image according to the operation step. The display 130 will display the real-time image and a plurality of lesions. The lesions include lung sliding (+), lung sliding (−), lung pulse, and lung point sign. The sign (+) indicates positive, and (−) indicates negative. In addition, the display 130 shows the patient's medical history and the complaints. The medical history and complaints in the examples include chest tightness, dyspnea, pneumothorax, and dry cough. If the diagnostic program 120 is still unable to give a proper diagnosis at this time, or the level of confidence has not reached a predetermined value, the diagnostic program 120 will load the operation steps corresponding to view 2. The user can scan the lungs at the indicated position of the thorax to obtain the real-time image. The display 130 will show the real-time image and a plurality of lesions. The lesions include A-line and B-line with both positive and negative options.

If the user did not observe lung sliding and the B-line in the image, after selecting the lung sliding and the B-line to be negative through the user interface 140, with the patient's complaints and medical history, the diagnostic program 120 can diagnose that the patient has 85% chance of having pneumothorax. If the user observed lung sliding and the B-line in the image, after selecting lung sliding and the B-line to be positive through the user interface 140, the diagnostic program 120 can diagnose that the patient has 75% chance of having an interstitial lung disease.

If the lesions found in view 2 are still insufficient to increase the level of confidence to the predetermined value, the diagnostic program 120 will load the next operation steps, which corresponds to view 3, and so on, until the diagnostic program 120 can obtain the sufficient level of confidence and derive the diagnosis.

In summary, the point-of-care ultrasound device and the diagnostic method provided by the embodiment can predefine which lesions may be found in each ultrasound view according to the POCUS related literature. Each ultrasound view can have different lesions and different definitions may be inputted in advance. By combining the patient's medical history, complaints, and lesions, the diagnostic program can arrive at the diagnosis. At the same time of scanning, each ultrasound view will allow the user to check whether the lesions are observed in this ultrasound view and the program would update the records. At the end of the diagnostic process, the POCUS will automatically generate several possible diagnoses for the user to determine the proper diagnosis. In this way, the diagnostic process will be relatively faster, and the patient can be treated more efficiently in the field of emergency medicine.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A diagnostic method using a point-of-care ultrasound device (POCUS), the method comprising:
storing at least one operation step corresponding to at least one organ, each operation step corresponding to an ultrasound view;
selecting a first operation step from the at least one operation step;
displaying a first ultrasound view corresponding to the first operation step;
scanning the at least one organ at a first position according to the first operation step to obtain a first real-time image, the first ultrasound view comprising the first real-time image and a plurality of first lesions;
selecting a first group of lesions from the plurality of first lesions; and
generating a first diagnosis according to at least the first group of lesions.

2. The method of claim 1, further comprising:
selecting a second operation step from the at least one operation step;
displaying a second ultrasound view corresponding to the second operation step;
scanning the at least one organ at a second position according to the second operation step, the second ultrasound view comprising a second real-time image and a plurality of second lesions, wherein the first position is different from the second position;
selecting a second group of lesions from the plurality of second lesions; and
generating a second diagnosis according to at least the first group of lesions and the second group of lesions.

3. The method of claim 1, further comprising:
displaying a plurality of conditions;
selecting at least one condition from the plurality of conditions; and
generating a third diagnosis according to at least the first group of lesions and the at least one condition.

4. The method of claim 3, wherein the plurality of conditions comprises a medical history and a patient complaint.

5. The method of claim 1, wherein the first diagnosis has a first level of confidence.

6. The method of claim 5, further comprising:
if the first level of confidence is higher than a first predetermined value, ending a diagnostic process.

7. The method of claim 5, further comprising:
if the first level of confidence is lower than a second predetermined value, selecting a second operation step from the at least one operation step and displaying a second ultrasound view corresponding to the second operation step;
scanning the at least one organ at a second position according to the second operation step, the second ultrasound view comprises a second real-time image and a plurality of second lesions, wherein the first position is different from the second position;
selecting a second group of lesions from the plurality of second lesions; and
generating a second diagnosis according to at least the first group of lesions and the second group of lesions, wherein the second diagnosis has a second level of confidence.

8. The method of claim 1, wherein an algorithm is used to generate the first diagnosis according to at least the first group of lesions, and the method further comprises optimizing the algorithm when the first diagnosis is confirmed or denied.

9. The method of claim 8, wherein:
the first diagnosis has a first level of confidence;
when the first diagnosis is confirmed, the algorithm is optimized to increase the level of confidence during a next diagnostic process; and
when the first diagnosis is denied, the algorithm is optimized to reduce the level of confidence during the next diagnostic process.

10. A point-of-care ultrasound device (POCUS) comprising:
a microcontroller configured to execute a diagnostic program, the diagnostic program comprising a plurality of operation steps each corresponding to an ultrasound view;
a probe configured to scan at least one organ at a first position to obtain a first real-time image when performing a first operation step of the plurality of operation steps;
a display coupled to the microcontroller, and configured to display a first ultrasound view corresponding to the first operation step, the first ultrasound view comprising the first real-time image and a plurality of first lesions; and
a user interface configured to select a first group of lesions from the plurality of first lesions;
wherein the microcontroller generates a first diagnosis according to at least the first group of lesions.

11. The POCUS of claim 10, wherein the diagnostic program further comprises an algorithm to generate the first diagnosis according to at least the first group of lesions, and when the first diagnosis is confirmed or denied, the algorithm is optimized.

12. The POCUS of claim 11, wherein:
the first diagnosis has a first level of confidence;
when the first diagnosis is confirmed, the algorithm is optimized to increase the level of confidence during a next diagnostic process;
when the first diagnosis is denied, the algorithm is optimized to reduce the level of confidence during the next diagnostic process.

13. The POCUS of claim 10, wherein:
the user interface provides the plurality of operation steps, the plurality of operation steps being selectable;
a second operation step is executed by the microcontroller for the probe to scan the at least one organ at a second position to obtain a second real-time image;
the display is further configured to display a second ultrasound view corresponding to the second operation step;
the second ultrasound view comprises the second real-time image and a plurality of second lesions;
a second group of lesions can be selected through the user interface from the plurality of second lesions; and
the microcontroller generates a second diagnosis according to at least the first group of lesions and the second group of lesions.

14. The POCUS of claim 10, wherein:
the display is a touch panel;
the plurality of first lesions are displayed on the touch panel; and
the first group of lesions is selected through the user interface.

15. A diagnostic method of using a point-of-care ultrasound device (POCUS), the method comprising:
storing a plurality of operation steps corresponding to at least one organ, each operation step corresponding to an ultrasound view;
selecting a first operation step from the plurality of operation steps;
displaying a first ultrasound view corresponding to the first operation step;
scanning the at least one organ at a first position according to the first operation step to obtain a first real-time image, the first ultrasound view comprising the first real-time image and a plurality of first lesions;
selecting a first group of lesions from the plurality of first lesions;
selecting a second operation step from the plurality of operation steps;
displaying a second ultrasound view corresponding to the second operation step;
scanning the at least one organ at a second position according to the second operation step to obtain a second real-time image, the second ultrasound view comprising the second real-time image and a plurality of second lesions;
selecting a second group of lesions from the plurality of second lesions; and
generating a plurality of first diagnoses according to at least the first group of lesions and the second group of lesions;
wherein the plurality of first diagnoses has a first level of confidence.

16. The method of claim 15 further comprising:
displaying a plurality of conditions;
selecting at least one condition from the plurality conditions; and
generating a plurality of second diagnoses according to the first group of lesions, the second group of lesions and the at least one condition, wherein the plurality of second diagnoses has a second level of confidence.

17. The method of claim 16, wherein the plurality of conditions comprises a medical history and a patient complaint.

18. The method of claim 15, wherein the first position is different from the second position.

19. The method of claim 15, wherein an algorithm is used to generate the plurality of first diagnoses according to at least the first group of lesions and the second group of lesions; and the method further comprises when a final diagnosis is selected from the plurality of first diagnoses, optimizing the algorithm.

20. The method of claim 19, wherein the final diagnosis has a level of confidence; when the final diagnosis is confirmed, the algorithm is optimized to increase the level of confidence of the final diagnosis during a next diagnostic process and to reduce the level of confidence of other diagnoses of the plurality of first diagnoses during their next diagnostic processes.
